(19) United States
(12) Patent Application Publication
(10) Pub. No.: US 2024/0398241 A1
(43) Pub. Date: Dec. 5, 2024

(54) Method and System for Resolving Respiratory Sinus Arrhythmia Aliasing

(71) Applicant: ChroniSense Medical Ltd., Yokneam Illit (IL)

(72) Inventors: Assaf Pressman, Pardes Hanna-Karkur (IL); Irina Karelin, Haifa (IL); Yelena Koren, Kiryat Bialik (IL); Din Hadass, Ramat Yishai (IL)

(21) Appl. No.: 18/328,616

(22) Filed: Jun. 2, 2023

(51) Int. Cl. A61B 5/0205; A61B 5/00

(52) U.S. Cl. CPC A61B 5/0205; A61B 5/7203; A61B 5/02416; A61B 5/0245; A61B 5/0816; A61B 5/1102

(57) ABSTRACT

A method for resolving sinus arrhythmia aliasing in a signal containing heartbeat and respiratory information is provided. The method determines a plurality of variable time intervals between successive heartbeats from a sensor providing a signal that includes heartbeat information. The sensors can generate signals from an electrocardiogram, a photoplethysmography, an echocardiographic, an impedance based assessment, and a ballistocardiography signal device. From the signal a respiratory sinus arrhythmia signal from the plurality of variable time intervals between successive heartbeats are generated. From this data a sinus arrhythmia periodogram is generated. A respiration rate from a respiration sensor providing a respiration signal containing a direct physical measurement of a person's respiration is generated, and a respiration rate is determined. The respiration sensor includes an accelerometer, a gyroscope, an air motion sensor, a microphone, an impedance based assessment, and a video camera. The respiration rate and heartbeat rate are used in resolving the aliasing in the sinus arrhythmia periodogram.

### CROSS-REFERENCE TO RELATED APPLICATIONS

None.

### FIELD

The present application relates to methods, systems, and devices that use signals that contain heart rate information and underlying respiratory information to determine respiration rates. The respiratory information is determined from the time variations between successive heartbeats. This time variation in the heart rate during a respiration cycle is referred to as respiratory sinus arrhythmia. However, there are problems in determining sinus arrhythmia if the heart rate is too slow or the respiration rate is too fast. Since the underlying respiratory information is sampled by measuring the time differences between successive heartbeats, this sampling (the time between heartbeats) is subject to the Nyquist theorem. If the time between successive heartbeats is greater than half the time between respirations, then aliasing of the underlying sinus arrhythmia signal occurs, and a determination of a respiration rate from the sinus arrhythmia is ambiguous. Specifically, this application relates to techniques to overcome the problem of respiratory sinus arrhythmia aliasing, which enables an improved measurement of respiration. Additionally, this application relates to using heartbeat and respiration information to compensate for inaccuracies in other medical parameter measurements.

### BACKGROUND

It should not be assumed that any of the approaches described in this section qualify as prior art merely by virtue of their inclusion in this section.

There are a number of signals that can be used to determine respiratory sinus arrhythmia. These include electrocardiogram (ECG), photoplethysmography (PPG), echocardiography, and ballistocardiography (BCG) signals. However, each of these signals suffers an aliasing problem when the time intervals between successive heartbeats are greater than half the time intervals between breaths. Said another way, the heart rate needs to be twice the respiration rate, or aliasing of the sinus arrhythmia will occur.

Aliasing occurs because each successive heartbeat samples the time variation between successive heartbeats. If the sampling does not meet the Nyquist sampling rate, aliasing occurs in the determination of the respiratory sinus arrhythmia. What is needed is methods and systems to resolve aliasing in the respiratory sinus arrhythmia signal. The following disclosure provides techniques and solutions to overcome the aliasing problem and provide an accurate respiration rate when aliasing occurs.

### SUMMARY

This summary is provided to introduce a selection of concepts in a simplified form that is further described below in the Detailed Description. This summary is not intended to identify key features or essential features of the claimed subject matter, nor is it intended to be used as an aid in determining the scope of the claimed subject matter.

Disclosed are methods, systems, and devices for overcoming sinus arrhythmia aliasing when processing a signal containing heartbeat and respiratory information. One aspect of the invention discloses a method for overcoming respiratory sinus arrhythmia aliasing using a respiration sensor in conjunction with the signal containing heartbeat and respiratory information. The method determines a plurality of times between successive heartbeats from a signal that includes heartbeat information and determines a heartbeat rate. The plurality of times represent sampling instants of the sinus arrhythmia signal. These times are interpolated to provide evenly-spaced sinus arrhythmia samples for spectral analysis algorithms. The variability in the plurality of times between successive heartbeats is used to generate a sampled sinus arrhythmia signal that can have aliasing. The signal can be from a sensor providing an electrocardiogram (ECG), a photoplethysmography (PPG), an echocardiographic, and a ballistocardiography signal. The respiration sensor can be one of an accelerometer, a gyroscope, an air motion sensor, a microphone, and a video camera.

From a respiration sensor, a respiration signal is generated that provides a direct physical measurement of respiration. This signal is generated in a manner that will not be aliased but can be less accurate than the respiration rate determined from the respiratory sinus arrhythmia.

From the sinus arrhythmia signal, a sinus arrhythmia periodogram is determined. A respiration rate is determined from a respiration sensor signal and used in association with the heartbeat rate to detect and resolve aliasing effects in the sinus arrhythmia periodogram.

Another aspect of the invention discloses a system for resolving respiratory sinus arrhythmia aliasing. The system includes a sensor for detecting heartbeats and generating a signal that includes heartbeat information. The system determines a plurality of variable time intervals between successive heartbeats from the signal and determines a heartbeat rate. The variability in the plurality of times between successive heartbeats is used to generate a respiratory sinus arrhythmia signal that can have aliasing.

The system includes a respiration sensor that generates a respiration signal which provides a direct physical measurement of respiration. This signal is generated in a manner that will not be aliased but can be less accurate than the respiration rate determined from the respiratory sinus arrhythmia.

The system processor then determines a sinus arrhythmia periodogram from the sampled sinus arrhythmia signal. The processer uses the determined respiration rate from a respiration sensor signal in association with the heartbeat rate to detect and resolve aliasing attributes in the sinus arrhythmia periodogram.

The sensors can include an electrocardiogram (ECG), a photoplethysmography (PPG), an echocardiographic, and a ballistocardiography sensor generating the associated signal. The respiration sensor can be one of an accelerometer, a gyroscope, an air motion sensor, a microphone, and a video camera.

Another aspect of the invention discloses a non-transitory computer-readable storage medium having embodied thereon instructions which, when executed by a processor, perform steps of a method resolving sinus arrhythmia aliasing in a signal containing heartbeat and respiratory information. The method includes determining a plurality of variable time intervals between successive heartbeats and a heartbeat rate from a sensor providing a signal that includes heartbeat information. From the plurality of variable time intervals between successive heartbeats, a sampled respiratory sinus arrhythmia signal is determined. Next, a respiration rate is determined from a respiration signal containing a direct physical measurement of a person's respiration. A sinus arrhythmia periodogram is generated from the sinus arrhythmia signal, and aliasing issues with the periodogram are resolved using the respiration rate in association with the heartbeat rate.

### BRIEF DESCRIPTION OF THE DRAWINGS

Embodiments are illustrated by way of example and not limitation in the figures of the accompanying drawings, in which like references indicate similar elements and in which:

FIG. 1A is a chart of an ECG, PPG, respiration state, and sensor information during a respiration cycle without aliasing.

FIG. 1B is a chart of an ECG, PPG, respiration state, and sensor information during a respiration cycle with aliasing.

FIG. 1C is a chart of the periodogram of the interpolated time between heartbeats without aliasing.

FIG. 1D is a chart of the periodogram of the interpolated heart sample data with aliasing.

FIG. 1E is a chart of the periodogram of the evenly spaced respiration sensor signal samples from a respiration indication sensor.

FIG. 2 is a block diagram showing an example system for determining and monitoring respiration rate using sinus arrhythmia and additional sensors.

FIG. 3 is a flow chart showing steps of an example method for resolving respiratory sinus arrhythmia aliasing.

FIG. 4A is a graph of a typical PPG signal.

FIG. 4B is a graph of a PPG signal affected by respiration cycles.

FIG. 5A is a graph of a periodogram and accelerometer of a PPG signal without aliasing at a high heart rate.

FIG. 5B is a graph of a periodogram and accelerometer of a PPG signal without aliasing at a slower heart rate.

FIG. 5C is a graph of a periodogram and accelerometer of a PPG signal with aliasing.

FIG. 6 is a block schematic diagram of an example computing device that can be utilized to implement aspects of the present technology.

### DETAILED DESCRIPTION

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical, and electrical changes can be made without departing from the scope of what is claimed. The following detailed description is, therefore, not to be taken in a limiting sense, and the scope is defined by the appended claims and their equivalents.

The present disclosure provides methods, systems, and devices for resolving respiratory sinus arrhythmia aliasing using other signals generated from other physical processes correlated with a person's respiration to resolve ambiguities that can occur in the sinus arrhythmia data. Further, the systems and methods disclosed can be used to overcome, correct, or calibrate inaccuracies in the detection and measurement of other physiological parameters. It will be understood that the scope of the invention is not limited by the examples provided herein and that features of one aspect or embodiment of the invention can be combined with a feature of a different aspect or aspects and/or embodiments of the invention.

Signals from different sources can be used to determine an accurate time between successive heartbeats. These signals need to include a distinctive marker during each heartbeat to accurately measure the time between each successive heartbeat. These signals include but are not limited to, electrocardiogram (ECG), photoplethysmography (PPG), echocardiographic, and ballistocardiography (BGC) signals.

An ECG signal contains a number of waveforms often referred to in the literature as waves. These ECG waveforms include the P, Q, R, S, and T waves, the R wave being the strongest signal and the RR time being the time between successive heartbeats. The RR times of successive heartbeats provide the strongest marker for measuring the variable time between heartbeats. Thus, the “R” peaks are the best candidate for determining the variation in the heart rate during a respiratory cycle and can be used in determining respiratory sinus arrhythmia. Additionally, the invention contemplates the use of other waves for measuring the time variations between successive heartbeats. This measurement could include the PP, QQ, SS, and TT times.

For a PPG signal, the amplitude of the optical signal increases and decreases during each heart pulse. This PPG signal provides a repetitive signal correlated with heartbeats. The optical peaks in the signal provide a distinctive marker that can be used to determine the time variation between successive heartbeats. This information can then be used to calculate the respiratory sinus arrhythmia based on the time variation of successive heartbeats.

Echocardiographic signals use high-frequency sound waves (ultrasound) to generate a picture or video of the heart activity. This video information can be processed to extract information on the time between successive heartbeats. The time variations between successive heartbeats can be used to determine a respiratory sinus arrhythmia signal.

Ballistocardiography is a noninvasive method based on the measurement of the body motion generated by the ejection of the blood at each cardiac cycle. This motion can be detected, and a signal is generated by direct contact or non-contact sensors. These sensors include but are not limited to a gyroscope, or accelerometer. The signal includes distinctive markers that can be extracted and used to determine the time variations between successive heartbeats, which in turn can be used to generate the respiratory cardiac arrhythmia signal. A person of ordinary skill in processing ballistocardiography signals would know how to extract the distinctive markers.

All of the signals mentioned above are subject to sinus arrhythmia aliasing when the heart rate is less than twice the respiratory rate. The cause of this aliasing is discussed in more detail below. To overcome or resolve the respiratory cardiac arrhythmia aliasing, additional information is required. The additional information can be respiratory rate information. This additional respiratory information can come from an additional sensor that samples a direct physical measurement associated with respiration. This additional respiratory information is used to detect and resolve aliasing artifacts in the generated respiratory sinus arrhythmia. Thus, the solutions provided below provide an accurate determination of the respiration rate during respiratory sinus arrhythmia aliasing.

### Respiratory Sinus Arrhythmia Aliasing

During a respiration cycle, a person's heart rate increases and decreases. The heart rate varies in a periodic pattern correlated with a respiration cycle. The heart rate increases as the person inhales and decreases as the person exhales. This variation in heart rate is referred to as respiratory sinus arrhythmia, and the variation in the heart rate can be used to determine a respiration rate. However, if the respiration rate is greater than one-half the heart rate, then aliasing will occur in the sampled respiratory sinus arrhythmia signal.

Aliasing occurs because each heartbeat is a sample of the variation of the time between successive heartbeat intervals, also referred to as sinus arrhythmia. If fewer than two successive samples (two heartbeats) are not taken during a breath cycle, then the underlying signal being sampled (the sinus arrhythmia) is distorted by the aliasing. When the sinus arrhythmia samples are transformed to the frequency domain, this distortion appears as new frequencies in the spectrum that were not in the sinus arrhythmia signal. The frequency spectrum of the sinus arrhythmia samples can be generated with a Discrete Fourier Transform (DFT) or preferably with a Fast Fourier Transform (FFT). Other transforms to determine the spectral content of the sampled sinus arrhythmia signal are contemplated and are generally referred to as periodograms which can include a data windowing function. When aliasing occurs, frequencies above the Nyquist frequency are folded around the Nyquist frequency, appearing as new frequencies below the Nyquist frequency. Here, the Nyquist frequency is half the heart rate which is the sampling rate.

### Variable Rate Sampling

Another attribute in generating a respiratory sinus arrhythmia periodogram is compensating for variable rate sampling of the sinus arrhythmia signal. Variable rate sampling is inherent in using the heartbeats for sampling successive heartbeat intervals. Because the time between heartbeats varies with each successive heartbeat, the sampling of the sinus arrhythmia does not occur at evenly spaced time intervals.

Algorithms, including the discrete Fourier transform and other periodogram algorithms, require evenly spaced samples to provide a meaningful output. Here, the variability of successive heartbeat intervals results in samples at varying times. To adjust the unevenly sampled data to be made compatible with frequency-transforming algorithms, the time-varying samples of successive heartbeat intervals need to be interpolated into evenly spaced intervals. Techniques for interpolation can be found in the document “Understanding the Lomb-Scargle Periodogram” by Jacob T. VanderPlas, The Astrophysical Journal Supplement Series, 236:16 (28 pp), 2018 May, https://doi.org/10.3847/1538-4365/aab76 which is incorporated by reference. Further, a person of ordinary skill in the art of digital signal processing would know how to transform the data into the frequency domain and interpolate variable rate data. Described more simply, the time variations between successive heartbeats are adjusted through interpolation to values that would have occurred if the samples were taken at evenly spaced times.

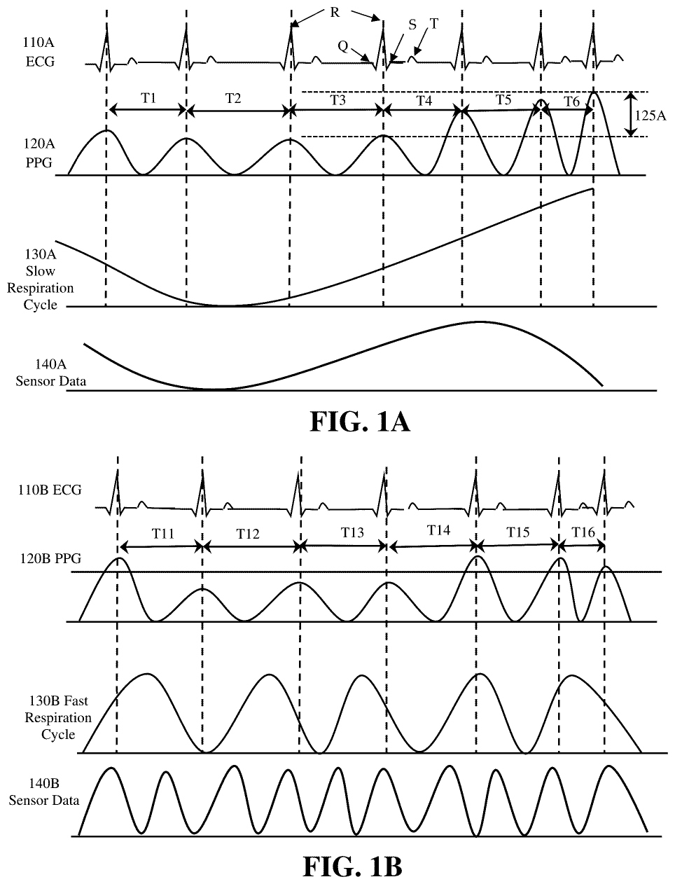

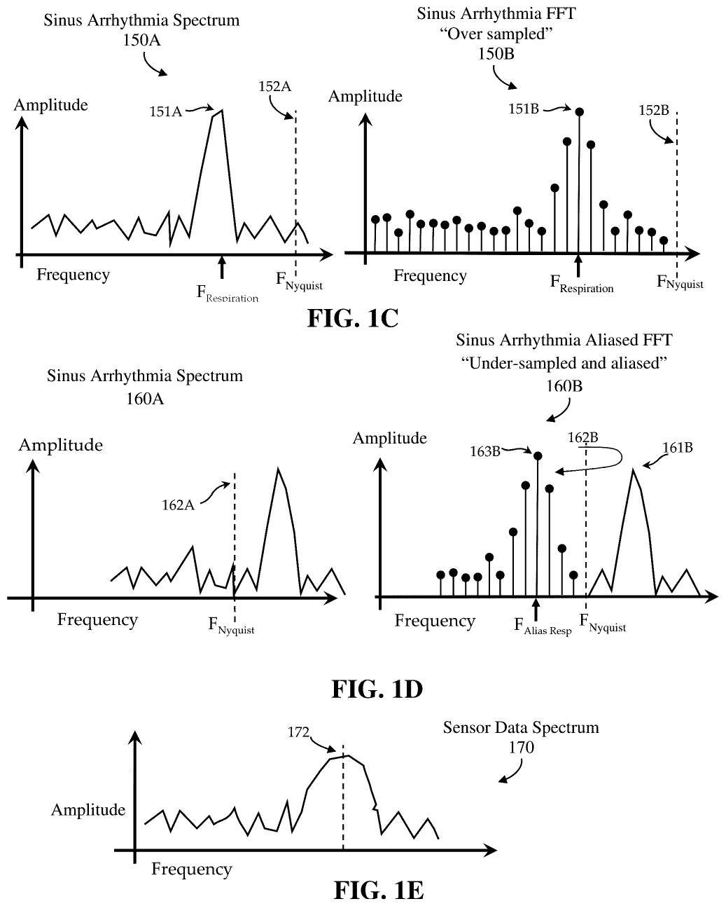

FIG. 1A is one example of a signal that includes variability of successive heartbeat intervals. In this figure, the marker for the heartbeat intervals is shown for the R peak to R peak times of an ECG signal 110A and for the optical peak-peak intervals shown in 120A for a PPG signal. However, this example is applicable to other sensors generating signal markers of successive heartbeat intervals, including an echocardiography sensor, and a ballistocardiography (BCG) signal.

An Electrocardiogram (ECG) signal 110A is shown where a person's respiration rate 130A is less than one-half the heart rate. The times T1-Tn are the times between heartbeats using the “RR” peaks. Between T1 and T2, the interval time increases as the heart rate decreases. This corresponds with a person exhaling or at the end of a respiration cycle. Then between T3 and T6, the interval times decrease as the heart rate increases during the inhalation segment of a respiration cycle. As can be seen in the ECG 110A signal, multiple heartbeats occur during the slow respiration cycle 130A, and thus aliasing does not occur when determining the variability of successive RR intervals.

A PPG signal 120A also includes heartbeat and respiratory information. As discussed above, it is a signal where respiratory sinus arrhythmia aliasing can occur. The PPG signal 120A represents the strength of an optical signal generated by a PPG sensor, which is correlated with the successive heartbeats with the optical peaks marking a heart pulse. While the PPG signal 120A optical peaks are shown to be aligned with the ECG signal 110A “R” peaks, there would normally be a temporal offset due to the time it takes a heart pulse to travel to the point where the PPG signal is measured.

Also, note that the amplitude 125A of the PPG signal 120A varies and is correlated with the respiration rate. This amplitude variation 125A can be used to determine a respiration rate. Thus, the PPG amplitude peaks are sampling not only the time variation of successive heartbeats but are also sampling the changes in the optical pulse amplitudes. Either of these can be used to generate a respiratory sinus arrhythmia signal, and both are subject to aliasing if the heart rate is not twice the respiratory rate.

Respiratory sensor data 140A is data of a physical attribute that is directly correlated with the respiration cycle. The sensor data can be an accelerometer or gyroscope that detects the movement of a person's chest. Also, the sensor data 140A can be a pressure device detecting the expansion of a person's chest or diaphragm. Further, the sensor data 140A can be from a microphone receiving the sound of a person breathing. Additionally, the sensor data 140A can be an airflow measuring device that detects airflow from the mouth or nostrils of the person being monitored or a video signal processed to extract a signal with a marker for each respiration cycle. Any sensor device that generates a signal correlated with the respiration rate can be used.

FIG. 1B illustrates signals that include heartbeat and respiratory information when the respiration rate is greater than one-half the heart rate and respiratory sinus arrhythmia aliasing occurs. The heart ECG signal 110B shows the “R” peaks. As shown, the respiration cycle is in the order of the heart rate. The time interval variations between the “R” peak intervals for successive heartbeat intervals represent the sampled sinus arrhythmia signal. These are time variations are shown by the intervals T11 . . . T16. As discussed above, the PPG signal 120B will also have a variation in the times between optical peaks for successive heartbeat intervals and will also exhibit amplitude variation in successive heartbeats. The respiration cycle 130B and the amplitude of the PPG signal 120B will vary and are correlated with the respiration cycle 130B. Sensor data 140B that directly represents the respiratory cycle is shown and can be generated by the devices discussed above.

Respiratory sinus arrhythmia aliasing occurs when the time intervals between heartbeats are greater than half a respiration cycle. For example, if the respiration cycle was twenty breaths per minute, then to determine the respiration rate without aliasing and using the sampled respiratory sinus arrhythmia signal, the heartbeat rate would have to be at least twice the respiration rate or forty heartbeats per minute. This is known as the Nyquist frequency.

Typically, to determine the respiration rate, a frequency analysis, such as a periodogram, is performed on the varying times between successive heartbeats. A periodogram is a way of representing frequency components in a data set and can be performed by a Fast Fourier Transform (FFT) along with different data windowing options. If the heart rate is too low or the respiration rate too fast, the Nyquist rate is violated, i.e., under-sampling. The result of the Nyquist violation is that the FFT will have artifacts of an aliased sampled sinus arrhythmia signal in the periodogram. The sampled sinus arrhythmia signal frequency components above the Nyquist frequency are folded around the Nyquist frequency and appear as aliased frequencies below the Nyquist frequency in the periodogram.

Referring to FIG. 1C, a Sinus Arrhythmia Spectrum 150A of the respiratory sinus arrhythmia signal is shown. A spectral peak 151A is shown at the respiration frequency. The Nyquist frequency 152A is shown for reference to the FFT spectrum 150B of the sampled data. The Sinus Arrhythmia FFT 150B can be other periodograms of the time interval variations between successive “R” peaks. A dominant peak 151B is shown, which corresponds to the respiration rate determined from the respiratory sinus arrhythmia. The Nyquist frequency 152B is shown above the respiration rate.

Referring to FIG. 1D, a Sinus Arrhythmia Spectrum 160A of respiratory sinus arrhythmia is shown. Spectrum 160A represents the underlying sinus arrhythmia signal without sampling. The Nyquist frequency 162A is shown for reference to the Sinus Arrhythmia Aliased FFT spectrum 160B of the sampled data.

The Sinus Arrhythmia Aliased FFT 160B shows the aliased spectrum when the heartbeat rate is less than twice the respiration rate. Because of the aliasing, the respiration rate spectrum 161B wraps around the Nyquist frequency 162B and generates an aliased respiration peak 163B in the FFT. To determine the actual respiratory rate utilizing the periodogram, additional information is required. This information can come from a signal representing a direct measurement of respiration. These direct measurements can be generated by respiration sensors, including but are not limited to an accelerometer, gyroscope, microphone, airflow sensor, or video camera. One skilled in the art of sensor data would know how to correlate the sampled sensor data with the associated respiratory cycle. Note, a FFT requires evenly spaced samples. Because the sampled successive heart intervals are not evenly spaced, caused by the variability in the heart rate due to sinus arrhythmia, the samples will require interpolation to generate evenly spaced samples for spectral analysis.

Referring to FIG. 1E, a periodogram 170 of the respiratory sensor signal is shown. The periodogram for the respiratory sensor, which can include an accelerometer, gyroscope, microphone, video camera, airflow sensor, or impedance change can have a broad spectrum with a mean 172 approximating the respiratory rate. As the spectrum is more broad, the accuracy of a respiratory rate evaluated from that spectrum can be low. This mean value can be used to determine if aliasing has occurred in the sinus arrhythmia spectrum. If the mean frequency 172 is above the Nyquist frequency 162B, one-half the heart rate, then aliasing occurs in the sinus arrhythmia signal.

### System for Overcoming Aliasing in Sinus Arrhythmia

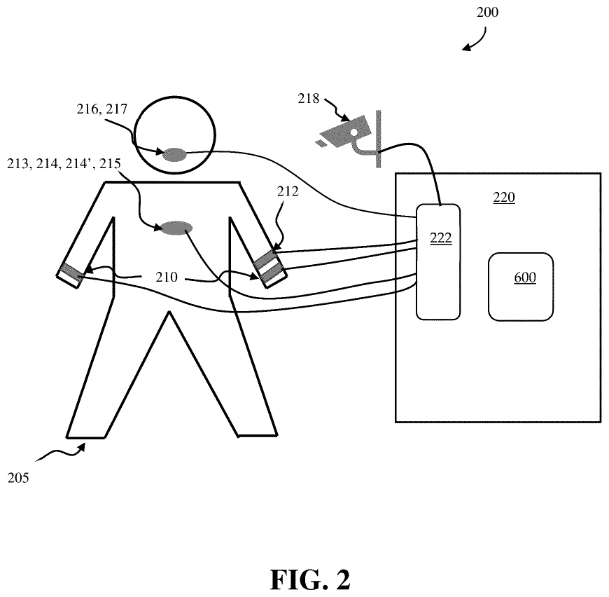

FIG. 2 is a block diagram illustrating components of a system 200 for resolving ambiguities in a sinus arrhythmia signal when aliasing occurs. The system is comprised of sensors 210, 212, 213, 214, 216, 217, and 218 that are adjacent to, coupled to, or near a person 205. Not all of the sensors are required for an operational system that can overcome aliasing. For a system that can overcome respiratory sinus arrhythmia aliasing, two sensors are required. A first sensor needs to generate a signal that includes heartbeat information, and a second sensor generates a signal that is directly correlated with a physical manifestation of respiration and not subject to aliasing, but might present less accuracy in assessment of respiration rate.

One system configuration uses an ECG sensor 210 to acquire a signal with heartbeat information. The signal includes heartbeat information from which variable time intervals between successive heartbeats are extracted. These times are used to generate a respiratory sinus arrhythmia signal. A processing system 220 detects the “R” peaks and generates the R-peat to R-peak times for successive heartbeats.

Another system configuration uses a PPG sensor 212 to acquire a signal with heartbeat information. The signal includes heartbeat information from the changes in the optical amplitude signal that corresponds to changes in blood flow and thus are correlated with a person's heartbeat. The times between the optical peaks are used to determine the variable time intervals between successive heartbeats. From this information, a respiratory sinus arrhythmia is extracted, and a sampled respiratory sinus arrhythmia signal is generated. The PPG sensor 212 can generate a PPG signal from a person's radial artery or finger. The PPG sensor 212 signal, as described above, can also have amplitude variations correlated with the respiratory cycle and can have baseline wandering correlated with the respiratory cycle.

The time variability between successive heartbeats can also be referred to as frequency modulation of the PPG signal pulsations due to the respiratory sinus arrhythmia heartbeats. Additionally, for the PPG signal, there is amplitude modulation of the optical signal peaks, and baseline wandering is due to mechanical changes in pressure on the heart by the rib cage.

In alternative system embodiments, an echocardiogram sensor 213 or a ballistocardiography (BCG) sensor 214′, 218 generates a signal that is used to determine the variable time intervals between successive heartbeats and from which the sampled respiratory sinus arrhythmia is determined. The echocardiogram sensor 213 is an ultrasound generation device and receiver from which a distinctive marker for each heartbeat can be generated.

The BCG sensor 214′, 218 measures the ballistic forces generated by the heart. The downward movement of blood through the descending aorta produces an upward recoil, moving the body upward with each heartbeat. As different parts of the aorta expand and contract, the body continues to move downward and upward in a repeating pattern. The BCG sensor 214′, 218 captures the repetitive motions of the human body, arising from the sudden ejection of blood into the great vessels with each heartbeat. The motions can be recorded by noninvasive methods from the surface of the body, such as an accelerometer 214′, or can be monitored using a video camera 218. Where a video camera 218 is used, the processing component 220 processes the image data from the video camera 218 to extract the motion associated with heartbeats. From the motions, the variability in time intervals between successive heartbeats can be determined to generate a sampled respiratory sinus arrhythmia signal. Note the same or different camera 218 can be used to monitor motions related to detecting respiration.

A second sensor is required for the above-described system embodiments to detect and resolve the aliasing that can occur in the generation of a sampled respiratory sinus arrhythmia signal. The second sensor is also referred to as a respiratory sensor. The respiratory sensor generates a signal that is a direct physical representation of the person's 205 respiration. The respiratory sensor signal needs to be sampled at a sufficient rate so that aliasing will not occur in the respiratory sensor data sampled from the respiratory sensor signal.

The accelerometer sensor 214 generates a signal based on the movement of the person's 205 chest because of the respiration cycle. This accelerometer sensor 214 can be located on the top of the chest, the side of the chest, on the back, or at any other point on a person's body where the motion is correlated with respiration. Another sensor is a gyroscope 215. While shown as being placed on the chest in FIG. 2, the gyroscope 215 can also be placed on the side of the chest or back.

The microphone sensor 216 generates an audio signal that can be processed in order to determine respiratory cycles. While the microphone sensor 216 is shown in FIG. 2 placed near the person's 205 mouth, the microphone sensor 216 can be placed elsewhere, including but not limited to the chest, throat, and on the person's 205 back.

The airflow sensor 217 generates a signal related to airflow into and out of the person's 205 mouth or nostrils. From the generated signal, times between breaths can be determined, and a respiration rate can be generated.

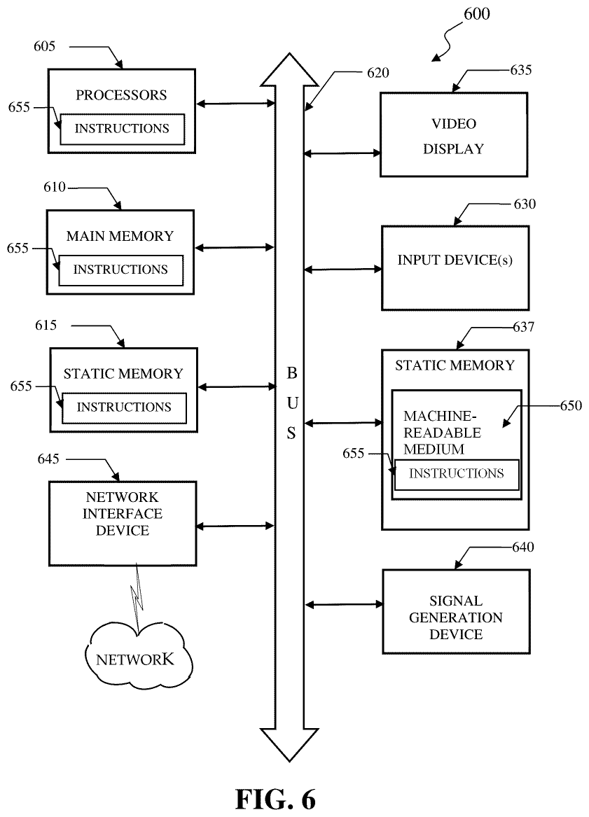

The sensor devices generate sensor signals that are connected to interface electronics component 222. This interface electronics component 222 contains any amplifiers, filters, and data sampling needed to provide sample data that can be processed by the processor 600. Details of the processor 600 are found below and illustrated in FIG. 6.

### Methods for Overcoming Respiratory Sinus Arrhythmia Aliasing

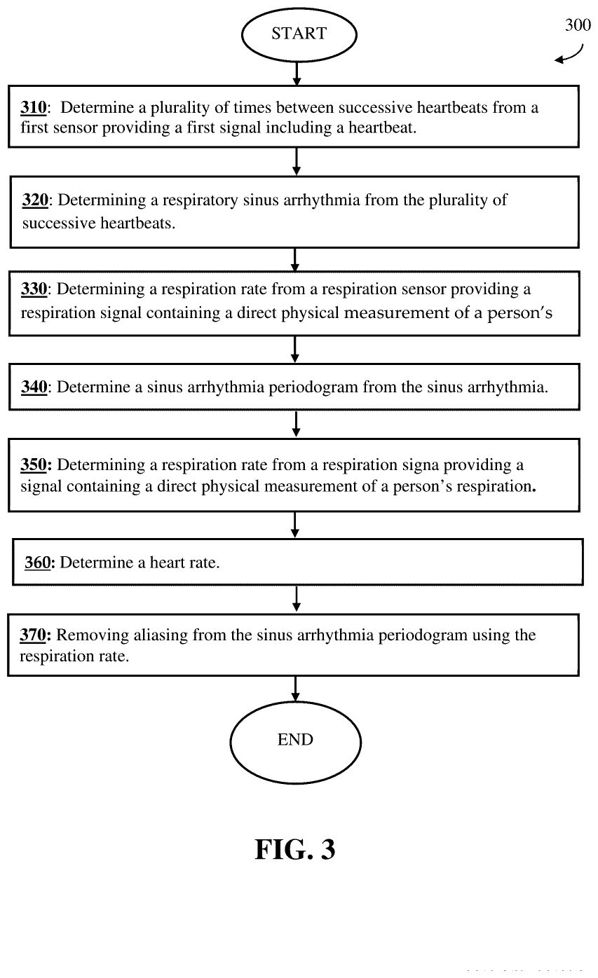

FIG. 3 shows a flow chart diagram showing an example method 300 for overcoming respiratory sinus arrhythmia aliasing. The method 300 utilizes a signal with heartbeat information that incorporates sampling of an underlying respiration signal and another signal that measures a physical attribute correlated with a person's respiration rate.

In step 310, a signal from a first sensor is processed to generate a plurality of times between successive heartbeats. The first sensor can generate an ECG signal, a PPG signal, or can generate a heartbeat signal derived from an echocardiographic or a ballistocardiograph signal. Each of these signals will include variations in the time between successive heartbeats resulting from respiratory sinus arrhythmia.

In step 320, a sampled respiratory sinus arrhythmia signal is generated from the plurality of times between successive heartbeats. The plurality of variable time intervals between successive heartbeats are not evenly spaced in time because the heartbeats are not evenly spaced in time. Thus, the sampling of the respiration rate is not evenly spaced in time. For this data to be useful for algorithms like the DFT, FFT, or other periodogram algorithms, the data samples need to be evenly spaced. This step can include the interpolation of the plurality of variable time intervals between successive heartbeats to generate a set of temporally evenly spaced variations of successive heartbeats.

In step 330, a respiration signal associated with a respiration sensor is processed to determine a respiration rate. The respiration signal can be generated by an accelerometer, a gyroscope, an air motion sensor, a microphone, and a video camera. The respiration signal needs to represent a direct physical attribute of respiration. An accelerometer or gyroscope signal should represent the motion of the chest or body caused by respiration. A microphone can generate an audio signal associated with respiration. The microphone can be directly in contact with the body or near the body. An airflow sensor can detect the movement of air from the mouth or nostrils or from a device that detects the use of air or oxygen being delivered to the mouth or nostrils. This movement of air is correlated with the respiration cycle and thus can be used to determine a respiration rate. The video camera can capture images of the chest expanding and contracting. From the image processing of the video signal, distinctive markers of the respiration cycle can be detected, and a respiration rate determined.

In step 340, the respiratory sinus arrhythmia periodogram is generated from the interpolated plurality of variable time intervals between successive heartbeats. The periodogram can use a DFT, a DFT with windowing, FFT, or any other suitable periodogram.

In step 350, a respiration rate is generated from the respiration signal samples. These samples are taken at a sampling rate where aliasing does not occur, but the signal can contain noise. For example, an accelerometer or gyroscope can pick up other body movements, which will degrade the signal and the estimate of the respiration rate. Thus, the estimated respiration rate will vary, or a spectrum of the data will exhibit a broad peak.

In step 350, a respiration rate is generated from the respiration signal samples. These samples are taken at a sampling rate where aliasing does not occur, but the signal can contain noise. For example, an accelerometer or gyroscope can pick up other body movements, which will degrade the signal and the estimate of the respiration rate. Thus, the estimated respiration rate will vary, or a spectrum of the data will exhibit a broad peak. The respiration rate from the respiration sensor can be determined in multiple ways. In one implementation, distinctive signal attributes can be counted over a fixed time interval. Another method for determining the respiration rate is to process the respiration data with a DFT, FFT, or other periodogram to extract the frequency components. The spectrum should have a peak at or near the respiration rate though the peak may be spread out across a range of frequencies because of noise. The mean value of the spread peak can be determined and taken as the respiration rate.

In step 360, a heartbeat rate is determined. The heartbeat rate can be determined from the plurality of variable time intervals between successive heartbeats. These variable time intervals are generated in step 310. Any suitable technique for determining the heartbeat rate can be used including counting the heartbeats in an interval or determining an FFT of the heartbeat data.

In step 370, the aliasing is detected and resolved by using the respiratory rate from the respiration sensor, the sinus arrhythmia periodogram and the heart rate information. Aliasing is detected when the respiration rate from the respiration sensor indicates a rate greater than half the detected heart rate. For example, if the heart rate is 50 beats per minute, the Nyquist frequency is 25 breaths per minute (bpm). If the respiratory sensor indicates a respiration rate of about 30 bpm, greater than the Nyquist frequency, then aliasing occurs in the sampled sinus arrhythmia signal and a peak in the sinus arrhythmia periodogram is an aliased respiration rate. The peak will be found at around 20 bpm. This is because a signal with a peak at 30 breaths per minute is aliased (folded) around the Nyquist frequency (25) and appears as a peak in the sinus arrhythmia periodogram at 20 bpm. Knowing the heart rate and thus the Nyquist frequency, the sinus arrhythmia respiration rate can be resolved by determining the amount of folding of the aliased sinus arrhythmia respiratory rate. A person of ordinary skill in the art of digital signal processing would be able to determine a method to resolve the sinus arrhythmia respiratory rate before aliasing.

### PPG Test Results

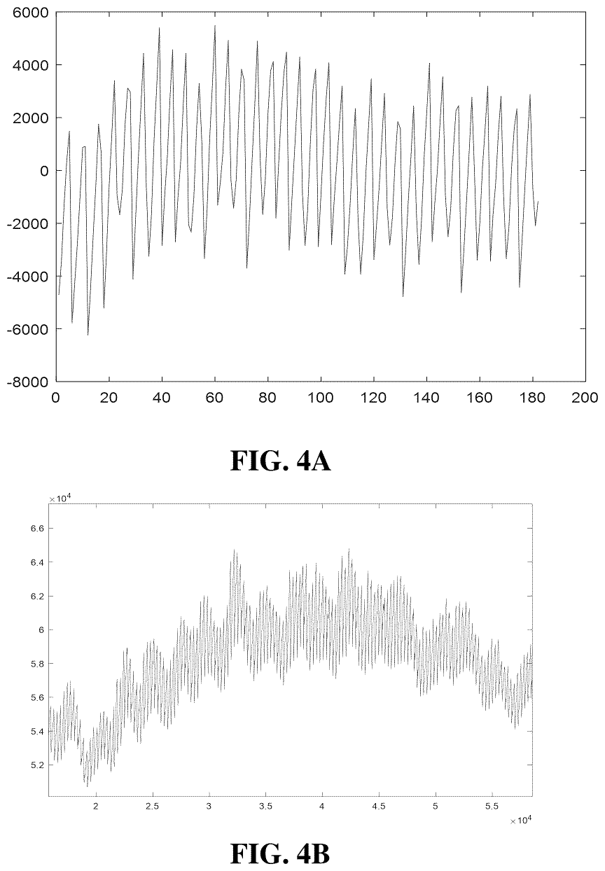

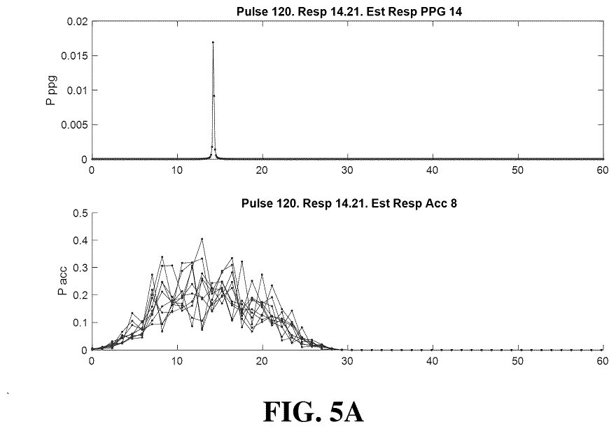

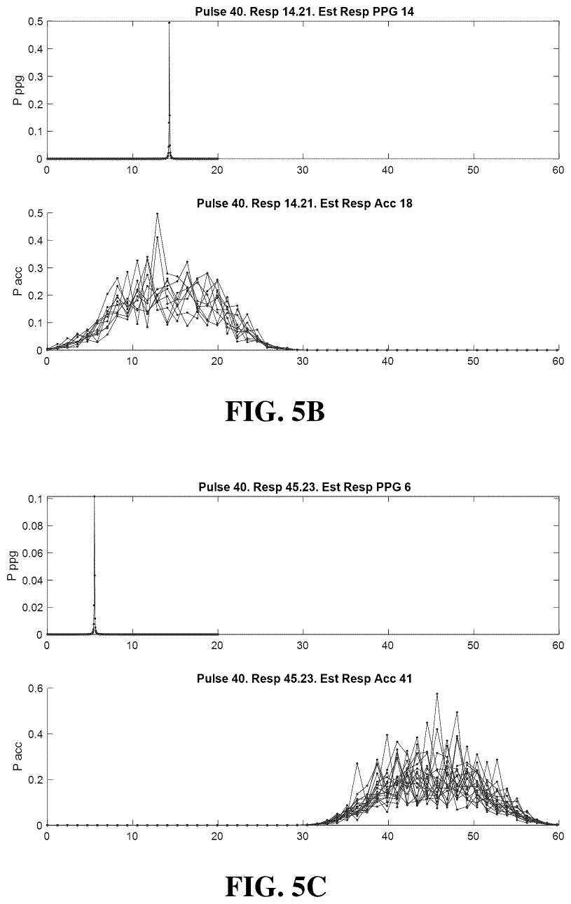

During a recent test, a simulation was run on PPG data from a PPG sensor coupled to a person. Additionally, an accelerometer was coupled to the person's wrist to provide a signal correlation with a physical manifestation of a person's respiration cycle. These recordings were performed at different respiration rates. The results of this test are shown in FIGS. 4A-4B and 5A-5C. Sinus arrhythmia aliasing is shown in FIG. 5C.

An example of the PPG signal is shown in FIGS. 4A and 4B. The heartbeats are indicated by peaks. The variations in the peak-to-peak times of successive heartbeats provide the data for determining respiratory sinus arrhythmia.

Normally, a person's respiration is between 5 to 10 breaths a minute, and the heart rate is between 60 to 80 beats per minute. In FIGS. 4A, 4B, and 5A, the heartbeat rate is significantly higher than the rate of respiration, and if heartbeats are used to sample the respiration signal, the Nyquist criteria is met.

When the respiration rate becomes faster (over half the heart rate), aliasing occurs in the sampled sinus arrhythmia signal. This results in frequencies above the Nyquist frequency being folded into frequencies below the Nyquist frequency. One skilled in the art of digital signal processing would be able to determine when aliasing occurs using heartbeat rate information and information from the respiration sensor. Additionally, one skilled in the art of digital signal processing would be able to unfold the aliased sinus arrhythmia respiration frequency in a periodogram using the heartbeat information and respiration signal frequency.

FIG. 5A shows the periodogram of the extracted respiratory sinus arrhythmia PPG signal at a high heart rate with respect to the periodogram of an accelerometer coupled to a person. As shown in FIG. 5A, the heart rate was one hundred and twenty (120) beats per minute, and the respiration rate was 14.21 breaths per minute.

Here, the periodogram from the accelerometer signal is prone to a large error (˜3 RPM RMS). In contrast, the respiratory sinus arrhythmia signal derived from the PPG signal shows higher accuracy determination of the respiration rate.

FIG. 5B shows the periodogram of the extracted respiratory sinus arrhythmia PPG signal at a lower heart rate with respect to the periodogram of an accelerometer coupled to a person. In this case, the heart rate was forty (40) beats per minute, and the respiration rate was 14.21 breaths per minute. Again, the accelerometer periodogram shows a large variance.

FIG. 5C shows the periodogram of the extracted respiratory sinus arrhythmia PPG signal at a heart rate where the respiratory sinus arrhythmia information is under-sampled and aliasing occurs. The heart rate was forty (40) beats per minute, and the respiration rate was 45.23 breaths per minute. The respiratory sinus arrhythmia signal derived from the PPG signal does not occur at that respiratory frequency but instead is aliased to 6 breaths per minute. Again, the accelerometer periodogram can have a large variance.

For all of these simulations shown in FIGS. 5B-5C, the following assumptions were made:
(1) The PPG signal is sampled by the heartbeat. The PPG signal is a single frequency signal.
(2) The accelerometer signal is not a single frequency due to the complicated mechanical mounting between the chest and the wrist. The signal is simulated by a narrow band random signal (±8 Hz) around the central frequency of respiration.
(3) The data is collected for 40 seconds.
(4) Ten iterations are presented due to the randomness explained in (2).

In one embodiment of the invention, an accelerometer is used to distinguish between the aliased peak and the correct peak in power spectra. One can use the PPG signal to determine the heart rate. These three parts are combined to produce an accurate measurement using the PPG signal while removing outliers using the accelerometer and ECG signal.

In one embodiment, the following algorithm can be used to resolve aliasing in the sinus arrhythmia algorithm. The algorithm utilizing the accelerometer and the PPG signal is shown below.
HR=Heart Rate determined from a PPG sensor.
ACCresprate=Accelerometer respiration rate.
PPGresprate=PPG respiration rate from derived respiratory sinus arrhythmia.

### Algorithm:

1. Estimate HR
2. If ACCresprate>HR/2 & PPGresprate<HR/2|If ACCresprate<HR/2 & PPGresprate>HR/2

```
If HR + PPGresprate = ACCresprate
  Report:
    HR + PPGresprate
else
  Report:
    PPGresprate
```

The accelerometer signal is sampled at a frequency higher than twice the fastest expected respiration rate. Thus, aliasing of the accelerometer signal will not occur. The limitation here is that the motion of the wrist does not necessarily track that of the chest, and therefore, the accuracy of the respiration rate derived from this signal may be low.

The current disclosure uses the best of both methods since it uses both signals to assess the respiration rate and uses both signals to resolve aliasing. More specifically, the respiration rate information comes from the PPG signal sampling the sinus arrhythmia signal, and the respiration sensor is used to detect and resolve the aliasing.

### Processing System

FIG. 6 is a diagrammatic representation of an example machine in the form of a computer system 600, within which a set of instructions for causing the machine to perform any one or more of the methodologies discussed herein may be executed and in conjunction with the discussed sensors. In various example embodiments, the machine operates as a standalone device or may be connected (e.g., networked) to other machines. In a networked deployment, the machine may operate in the capacity of a server or a client machine in a server-client network environment or as a peer machine in a peer-to-peer (or distributed) network environment. The machine may be, for example, a base station, a personal computer (PC), a tablet PC, a personal digital assistant (PDA), a cellular telephone, a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 600 includes a processor or multiple processors 605 (e.g., a central processing unit (CPU), a graphics processing unit (GPU), Digital Signal Processor, Neural Processor Unit (NPU) or any combination thereof), and a main memory 610 and static memory 615, which communicate with each other via a bus 620. The computer system 600 may further include a video display 635 (e.g., a liquid crystal display (LCD)). The computer system 600 may also include an alpha-numeric input device(s) 630 (e.g., a keyboard), a cursor control device (e.g., a mouse), a voice recognition or biometric verification unit (not shown), a drive unit 637 (also referred to as disk drive unit), a signal generation device 640 (e.g., a speaker), and a network interface device 645. The computer system 600 may further include a data encryption module (not shown) to encrypt data.

The drive unit 637 includes a computer or machine-readable medium 650 on which is stored one or more sets of instructions and data structures (e.g., instructions 655) embodying or utilizing any one or more of the methodologies or functions described herein. The instructions 655 may also reside, completely or at least partially, within the main memory 610 and/or within static memory 615 and/or within the processors 605 during execution thereof by the computer system 600. The main memory 610, static memory 615, and the processors 605 may also constitute machine-readable media.

The instructions 655 may further be transmitted or received over a network via the network interface device 645 utilizing any one of a number of well-known transfer protocols (e.g., Hyper Text Transfer Protocol (HTTP)). While the machine-readable medium 650 is shown in an example embodiment to be a single medium, the term “computer-readable medium” should be taken to include a single medium or multiple media (e.g., a centralized or distributed database and/or associated caches and servers) that store the one or more sets of instructions. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, and carrier wave signals. Such media may also include, without limitation, hard disks, flash memory cards, digital video disks, random access memory (RAM), read-only memory (ROM), and the like. The example embodiments described herein may be implemented in an operating environment comprising software installed on a computer, in hardware, or in a combination of software and hardware.

Not all components of the computer system 600 are required and thus portions of the computer system 600 can be removed if not needed, such as Input/Output (I/O) devices (e.g., input device(s) 630). One skilled in the art will recognize that the Internet service may be configured to provide Internet access to one or more computing devices that are coupled to the Internet service and that the computing devices may include one or more processors, buses, memory devices, display devices, input/output devices, and the like. Furthermore, those skilled in the art may appreciate that the Internet service may be coupled to one or more databases, repositories, servers, and the like, which may be utilized in order to implement any of the embodiments of the disclosure as described herein.

The corresponding structures, materials, acts, and equivalents of all means or step plus function elements in the claims below are intended to include any structure, material, or act for performing the function in combination with other claimed elements as specifically claimed. The description of the present technology has been presented for purposes of illustration and description but is not intended to be exhaustive or limited to the present technology in the form disclosed. Many modifications and variations will be apparent to those of ordinary skill in the art without departing from the scope and spirit of the present technology. Exemplary embodiments were chosen and described in order to best explain the principles of the present technology and its practical application, and to enable others of ordinary skill in the art to understand the present technology for various embodiments with various modifications as are suited to the particular use contemplated.

Aspects of the present technology are described above with reference to flowchart illustrations and/or block diagrams of methods, apparatus (systems) and computer program products according to embodiments of the present technology. It will be understood that each block of the flowchart illustrations and/or block diagrams, and combinations of blocks in the flowchart illustrations and/or block diagrams can be implemented by computer program instructions. These computer program instructions may be provided to a processor of a general-purpose computer, special-purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable medium that can direct a computer, other programmable data processing apparatus, or other devices to function in a particular manner, such that the instructions stored in the computer-readable medium produce an article of manufacture, including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods, and computer program products according to various embodiments of the present technology. In this regard, each block in the flowchart or block diagrams may represent a module, section, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or combinations of special purpose hardware and computer instructions.

In the following description, for purposes of explanation and not limitation, specific details are set forth, such as particular embodiments, procedures, techniques, etc., in order to provide a thorough understanding of the present invention. However, it will be apparent to one skilled in the art that the present invention may be practiced in other embodiments that depart from these specific details.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments. Furthermore, depending on the context of discussion herein, a singular term may include its plural forms, and a plural term may include its singular form. Similarly, a hyphenated term (e.g., “on-demand”) may be occasionally interchangeably used with its non-hyphenated version (e.g., “on demand”), a capitalized entry (e.g., “Software”) may be interchangeably used with its non-capitalized version (e.g., “software”), a plural term may be indicated with or without an apostrophe (e.g., PE's or PEs), and an italicized term (e.g., “N+1”) may be interchangeably used with its non-italicized version (e.g., “N+1”). Such occasional interchangeable uses shall not be considered inconsistent with each other.

Also, some embodiments may be described in terms of “means for” performing a task or set of tasks. It will be understood that a “means for” may be expressed herein in terms of a structure, such as a processor, a memory, an I/O device such as a video camera, or combinations thereof. Alternatively, the “means for” may include an algorithm that is descriptive of a function or method step, while in yet other embodiments the “means for” is expressed in terms of a mathematical formula, prose, or as a flow chart or signal diagram.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It is noted that the terms “coupled,” “connected,” “connecting,” “electrically connected,” etc., are used interchangeably herein to generally refer to the condition of being electrically/electronically connected. Similarly, a first entity is considered to be in “communication” with a second entity (or entities) when the first entity electrically sends and/or receives (whether through wireline or wireless means) information signals (whether containing data information or non-data/control information) to the second entity regardless of the type (analog or digital) of those signals. It is further noted that various figures (including component diagrams) shown and discussed herein are for illustrative purpose only and are not drawn to scale.

If any disclosures are incorporated herein by reference and such incorporated disclosures conflict in part and/or in whole with the present disclosure, then to the extent of conflict, and/or broader disclosure, and/or a broader definition of terms, the present disclosure controls. If such incorporated disclosures conflict in part and/or in whole with one another, then to the extent of conflict, the later-dated disclosure controls.

While various embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the invention as defined by the appended claims and otherwise appreciated by one of ordinary skill in the art. Thus, the breadth and scope of a preferred embodiment should not be limited by any of the above-described exemplary embodiments.

What is claimed is:

1. A method for resolving sinus arrhythmia aliasing in a signal containing heartbeat and respiratory information, the method comprising:
determining a plurality of variable time intervals between successive heartbeats from a sensor providing a signal that includes heartbeat information;
determining a respiratory sinus arrhythmia signal from the plurality of variable time intervals between successive heartbeats;
determining a respiration rate from a respiration sensor, the respiration sensor providing a respiration signal containing a direct physical measurement of a person's respiration;
determining a sinus arrhythmia periodogram from the sinus arrhythmia signal;
determining a heartbeat rate from the plurality of variable time between successive heartbeats; and
resolving aliasing in the sinus arrhythmia periodogram using the respiration rate and the heartbeat rate.

2. The method of claim 1, wherein the signal containing heartbeat and respiratory information is from one of an electrocardiogram (ECG), a photoplethysmography (PPG), an echocardiogram, and a ballistocardiography signal.

3. The method of claim 1, wherein the respiration signal containing the direct physical measurement of respiration is from one of an accelerometer, a gyroscope, an air motion sensor, a microphone, and a video camera.

4. The method of claim 1, wherein the determining the respiratory sinus arrhythmia includes interpolating the plurality of successive heartbeats, thereby generating a plurality of equally spaced successive heartbeats.

5. The method of claim 1, wherein the determining the respiration rate includes generating a respiration periodogram, the respiration periodogram having a mean frequency.

6. The method of claim 1, wherein the signal containing heartbeat and respiratory information is an electrocardiogram (ECG) signal.

7. The method of claim 1, wherein the signal containing heartbeat and respiratory information is a photoplethysmography (PPG) signal.

8. A system for resolving sinus arrhythmia aliasing in a signal containing heartbeat and respiratory information:
a heartbeat sensor configured to generate a plurality of variable time intervals between successive heartbeats from a signal including heartbeat information;
a respiration sensor for generating a plurality of respiration samples;
a processor configured to execute the steps:
determining a respiratory sinus arrhythmia signal from the plurality of variable time intervals between successive heartbeats;
determining a respiration rate from a respiration sensor providing a respiration signal containing a direct physical measurement of a person's respiration;
determining a sinus arrhythmia periodogram from the sinus arrhythmia signal;
determining a heartbeat rate from the plurality of variable time between successive heartbeats; and
resolving aliasing from the sinus arrhythmia periodogram using the respiration rate and the heartbeat rate.

9. The system of claim 8, wherein the signal containing heartbeat and respiratory information is from one of, an electrocardiogram (ECG), a photoplethysmography (PPG), an echocardiogram, and a ballistocardiography signal.

10. The system of claim 8, wherein the respiration signal containing the direct physical measurement of respiration is from one of an accelerometer, a gyroscope, an air motion sensor, a microphone, and a video camera.

11. The system of claim 8, wherein the determining the respiratory sinus arrhythmia includes interpolating the plurality of successive heartbeats, thereby generating a plurality of equally spaced successive heartbeats.

12. The system of claim 8, wherein the determining the respiration rate includes generating a respiration periodogram, the respiration periodogram having a mean frequency.

13. The system of claim 8, wherein the signal containing heartbeat and respiratory information is an electrocardiogram (ECG) signal.

14. The method of claim 8, wherein the signal containing heartbeat and respiratory information is a photoplethysmography (PPG) signal.

15. A non-transitory computer-readable storage medium having embodied thereon instructions, which when executed by a processor, perform steps of a method resolving sinus arrhythmia aliasing in a signal containing heartbeat and respiratory information, the method comprising:
determining a plurality of variable time intervals between successive heartbeats from a sensor providing a signal that includes heartbeat information;
determining a respiratory sinus arrhythmia signal from the plurality of variable time intervals between successive heartbeats;
determining a respiration rate from a respiration sensor providing a respiration signal containing a direct physical measurement of a person's respiration;
determining a sinus arrhythmia periodogram from the sinus arrhythmia signal;
determining a heartbeat rate from the plurality of variable time between successive heartbeats; and
resolving aliasing in the sinus arrhythmia periodogram using the respiration rate and the heartbeat rate.

16. The non-transitory computer-readable storage medium of claim 15, wherein the signal containing heartbeat and respiratory information is from one of, an electrocardiogram (ECG), a photoplethysmography (PPG), an echocardiographic, and a ballistocardiography signal.

17. The non-transitory computer-readable storage medium of claim 15, wherein the respiration signal containing the direct physical measurement of respiration is from one of an accelerometer, a gyroscope, an air motion sensor, a microphone, and a video camera.

18. The non-transitory computer-readable storage medium of claim 15, wherein the determining the respiratory sinus arrhythmia includes interpolating the plurality of successive heartbeats, thereby generating a plurality of equally spaced successive heartbeats.

19. The non-transitory computer-readable storage medium of claim 15, wherein the determining the respiration rate includes generating a respiration periodogram, the respiration periodogram having a mean frequency.

20. The non-transitory computer-readable storage medium of claim 15, wherein the signal containing heartbeat and respiratory information is an electrocardiogram (ECG).
